(19) United States
(12) Patent Application Publication
(10) Pub. No.: US 2011/0034812 A1
(43) Pub. Date: Feb. 10, 2011

(54) PULMONARY ARTERY PRESSURE BASED SYSTOLIC TIMING INTERVALS AS A MEASURE OF RIGHT VENTRICULAR SYSTOLIC PERFORMANCE

(76) Inventors: Abhilash Patangay, Inver Grove Heights, MN (US); Ramesh Wariar, Blaine, MN (US); Jon Peterson, Mahtomedi, MN (US)

Correspondence Address: SCHWEGMAN, LUNDBERG & WOESSNER/BSC-CRM, PO BOX 2938, MINNEAPOLIS, MN, 55402, US

(21) Appl. No.: 12/841,782

(22) Filed: Jul. 22, 2010

Related U.S. Application Data
(60) Provisional application No. 61/232,662, filed on Aug. 10, 2009.

(51) Int. Cl. A61B 5/021; A61N 1/365

(52) U.S. Cl. 600/485; 607/23

(57) ABSTRACT

Systems and methods include identifying a first portion and a second portion of a pulmonary artery pressure (PAP) signal during a cardiac cycle. A first timing interval between the first portion and the second portion is obtained and data related to the first timing interval is trended to provide a chronic physiological prognostic indicator. In an embodiment, a second timing interval is obtained from a third portion and a fourth portion of the PAP signal. Then, a function of the first and second timing intervals is trended to provide the chronic physiological prognostic indicator. In one instance, a ratio of the first interval to the second interval is calculated to provide an estimated right ventricle ejection fraction (RVEF) and the RVEF is trended.

### CROSS-REFERENCE TO RELATED APPLICATION

This application claims the benefit of U.S. Provisional Application No. 61/232,662, filed on Aug. 10, 2009, under 35 U.S.C. §119(e), the benefit of priority of which is claimed herein, and which is incorporated herein by reference in its entirety.

### TECHNICAL FIELD

This document pertains generally to implantable medical devices, and more particularly, but not by way of limitation, to using systolic timing intervals to measure ventricular performance.

### BACKGROUND

Heart disease (cardiomyopathy) can cause a patient to exhibit symptoms of congestive heart failure (CHF). CHF is a result of the weakening of the heart's cardiac function characterized by reduced pumping capacity and efficiency. Chronic cardiac rhythm problems can also be the result of cardiomyopathy. The modification of the heart's structure that causes the reduction in pumping capacity may also result in changes in the heart's electrical characteristics. For instance, the heart's electrical pathways can become stretched out of shape and chemically damaged. These changes may make arrhythmias more likely in CHF patients.

Implantation of a pacemaker is one method of treatment for arrhythmias in CHF patients. Although many types of heart problems may require a pacemaker, cardiac resynchronization therapy (CRT) is a treatment suited for CHF patients. CRT uses a pacemaker with multiple pacing leads to coordinate the heart's chambers to act together in a sequence to efficiently pump blood.

It is likely that CRT candidates will have various forms of cardiomyopathy, and these patients may exhibit other measurable symptoms of reduced cardiac function besides arrhythmia. The reduced cardiac function of the heart is taken into account when applying CRT in order to tailor the treatment based on the needs of a particular patient. Various external factors may also be taken into account by the pacing system, such as the state of the current activity of the patient.

Rate adaptive pacemakers can estimate body activity by detecting body movements or breathing rate and depth. Using the estimated body activity level, pacing rates can be adaptively modified and applied to the heart. Indicators such as body activity can provide a rough estimate of metabolic demand for a given patient.

### OVERVIEW

It would be beneficial to have more accurate measures of metabolic demand, especially measures that can determine the pumping capacity and pumping efficiency of a heart in order to measure and improve the efficacy of the therapy for a CHF patient. Right ventricular function can be shown to be a significant prognostic factor in CHF patients. One measure of ventricular function is the right ventricular ejection fraction (RVEF), which is the fraction of blood pumped out of the right ventricle in a contraction. Pulmonary artery pressure (PAP) is related to right ventricle function and can be used to derive diagnostic and prognostic indications.

In normal individuals the right ventricle (RV) functions as a priming pump for the left ventricle. Right ventricle dysfunction is associated with poor prognosis in heart failure (HF) patients. Previously, there has not a good way to measure and trend RV function in patients. Traditional RVEF measurement is cumbersome and difficult because RV geometry is complex. Right ventricle systolic timing intervals can be related to RV cardiac pump function, but this measurement can require an ECG and a PAP tracing Like RVEF such measurements are cumbersome and are not chronically available in patients.

In patients with a pacemaker/CRT device and a pulmonary artery pressure sensor, a combination of the two measurement mechanisms enables the measurement of RV systolic timing intervals. Thus, using this combination allows chronic trending of RV systolic timing intervals and RV function in HF patients.

Example 1 describes a system comprising a machine-readable media and one or more processors communicatively coupled to the machine-readable media. The machine-readable media can include instructions, which when executed by the one or more processors, cause the one or more processors to identify a first portion of a pulmonary artery pressure (PAP) signal during a cardiac cycle. The one or more processors can then identify a second portion of the PAP signal during the cardiac cycle, obtain a first timing interval between the first portion and the second portion, and then trend data related to the first timing interval to provide a chronic physiological prognostic indicator.

In Example 2, the system of Example 1 is optionally configured to identify a feature of an intrinsic electrical cardiac signal during the cardiac cycle and correlate the feature of the intrinsic electrical cardiac signal with a position on the PAP signal to identify the first portion of the PAP signal.

In Example 3, the systems of Example 1 or 2 are optionally configured to identify a feature indicative of the start of depolarization of ventricles.

In Example 4, the systems of any one or more of Examples 1-3 optionally configured to identify a start of a right ventricle pre-ejection period and identify an end of the right ventricle pre-ejection period.

In Example 5, the system of any one or more of Examples 1-4 are optionally configured to identify a start of a right ventricle ejection time and identify an end of the right ventricle ejection time.

In Example 6, the system of any one or more of Examples 1-5 are optionally configured to identify a third portion of the PAP signal during the cardiac cycle; identify a fourth portion of the PAP signal during the cardiac cycle; and obtain a second timing interval between the third portion and the fourth portion. The system is also configured to trend a function of the first and second timing intervals to provide the chronic physiological prognostic indicator.

In Example 7, the system of any one or more of Examples 1-6 are optionally configured to calculate a ratio of the first interval to the second interval to provide an estimated right ventricle ejection fraction (RVEF) and trend the estimated RVEF.

In Example 8, the system of any one or more of Examples 1-7 are optionally configured to compare the chronic physiological prognostic indicator to a threshold value to provide a result and identify a treatment in response to the result.

In Example 9, the system of any one or more of Examples 1-8 are optionally configured to obtain a plurality of chronic physiological prognostic indicators over time; trend the plurality of chronic physiological prognostic indicators to provide a result; and identify a treatment in response to the result.

In Example 10, the system of any one or more of Examples 1-9 are optionally configured to determine systolic function based on the chronic physiological prognostic indicator; determine whether the systolic function is improving; and adjust one or more pacing parameters when the systolic function is not improving, wherein the pacing parameters are to configure an implanted cardiac device.

In Example 11, the system of any one or more of Examples 1-10 are optionally configured comprising a chronically implanted device having one or more sensors to acquire the PAP signal during the cardiac cycle.

Example 12 describes a method comprising: identifying a first portion of a pulmonary artery pressure (PAP) signal during a cardiac cycle; identifying a second portion of the PAP signal during the cardiac cycle; obtaining a first timing interval between the first portion and the second portion; and trending data related to the first timing interval to provide a chronic physiological prognostic indicator.

In Example 13, the method of Example 12 is optionally performed such that identifying the first portion of the PAP signal during the cardiac cycle comprises: identifying a feature of an intrinsic electrical cardiac signal during the cardiac cycle; and correlating the feature of the intrinsic electrical cardiac signal with a position on the PAP signal to identify the first portion of the PAP signal

In Example 14, the methods of Examples 12 or 13 are optionally performed such that identifying the feature of the intrinsic electrical cardiac signal during the cardiac cycle comprises identifying a feature indicative of the start of depolarization of ventricles.

In Example 15, the methods of any one or more of Examples 12-14 are optionally performed such that identifying the first portion of the PAP signal during the cardiac cycle comprises identifying a start of a right ventricle pre-ejection period and such that identifying the second portion of the PAP signal during the cardiac cycle comprises identifying an end of the right ventricle pre-ejection period.

In Example 16, the methods of any one or more of Examples 12-15 are optionally performed such that identifying the first portion of the PAP signal during the cardiac cycle comprises identifying a start of a right ventricle ejection time, and such that identifying the second portion of the PAP signal during the cardiac cycle comprises identifying an end of the right ventricle ejection time.

In Example 17, the methods of any one or more of Examples 12-16 are optionally performed comprising: identifying a third portion of the PAP signal during the cardiac cycle; identifying a fourth portion of the PAP signal during the cardiac cycle; and obtaining a second timing interval between the third portion and the fourth portion. In addition, the methods of any one or more of Examples 10-14 are optionally performed such that trending data related to the timing interval comprises trending a function of the first and second timing intervals to provide the chronic physiological prognostic indicator.

In Example 18, the methods of any one or more of Examples 12-17 are optionally performed such that trending the function of the first and second timing intervals comprises: calculating a ratio of the first interval to the second interval to provide an estimated right ventricle ejection fraction (RVEF); and trending the estimated RVEF.

In Example 19, the methods of any one or more of Examples 12-18 are optionally performed comprising: comparing the chronic physiological prognostic indicator to a threshold value to provide a result; and identifying a treatment in response to the result.

Example 20 describes a system comprising: a physiological sensor to obtain a pulmonary artery pressure (PAP) signal; a memory device to store the PAP signal; means for identifying a first portion of the PAP signal during a cardiac cycle; means for identifying a second portion of the PAP signal during the cardiac cycle; means for obtaining a first timing interval between the first portion and the second portion; and means for trending data related to the first timing interval to provide a chronic physiological prognostic indicator.

Example 21 describes a machine-readable medium including instructions, which when executed on a machine, cause the machine to: identify a first portion of a pulmonary artery pressure (PAP) signal during a cardiac cycle; identify a second portion of the PAP signal during the cardiac cycle; obtain a first timing interval between the first portion and the second portion; and trend data related to the first timing interval to provide a chronic physiological prognostic indicator.

This overview is intended to provide an overview of subject matter of the present patent application. It is not intended to provide an exclusive or exhaustive explanation. The Detailed Description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. Some embodiments are illustrated by way of example, and not limitation, in the figures of the accompanying drawings in which:

FIG. 1 is a schematic diagram illustrating a system that enables physician-patient communication;

FIG. 2 is a schematic diagram illustrating an implanted medical device system;

FIG. 3 is a block diagram illustrating an implantable medical device;

FIG. 4 is a flow chart illustrating a method for obtaining a prognostic indicator;

FIG. 5 is an illustration of three signals;

FIG. 6 is a flow chart illustrating a method for managing pacing parameters;

FIG. 7 is a flow chart illustrating a method for patient management; and

FIG. 8 is a block diagram illustrating a machine in the example form of a computer system, within which a set or sequence of instructions for causing the machine to perform any one of the methodologies discussed herein may be executed, according to various embodiments.

### DETAILED DESCRIPTION

The examples described herein include systems and methods for measuring right ventricular systolic performance. It can be shown that right ventricular performance, as measured by right ventricular systolic time intervals (STIs), have a correlation to pulmonary hemodynamic parameters. Some STIs of interest include, but are not limited to, the right ventricular preejection period (RVPEP) and the right ventricular ejection time (RVET). The ratio of the RVPEP and the RVET provide a right ventricular ejection fraction (RVEF). Right ventricular STIs can be measured using one or more signals, including an electrocardiogram, a phonocardiogram, central venous pressure, carotid pulse, intracardiac impedance, or a pulmonary artery pressure curve.

The RVPEP can be taken as the time lapsed from the onset of electrical systole to the opening of the semilunar valve. RVPEP can also be considered the interval from the onset of the ventricular depolarization to the beginning of ejection—e.g., an electrical start to a mechanical start in a cardiac cycle. The RVET can be taken as the period during which the pulmonary semilunar valve remains open. The RVEF represents the amount of blood ejected from the right ventricle or the stroke volume (end-systolic volume minus the end-diastolic volume) divided by the end-diastolic volume. The RVEF can be estimated from the ratio of the RVPEP and the RVET.

Right ventricular STIs either alone or in combination can be used to assess right ventricular function. For heart failure patients, right ventricular dysfunction can be used as a short-term prognostic indicator. As an example, RVEF can be used to stratify patients with CHF who have similarly depressed left ventricular ejection fractions (LVEF) and ultimately provide a better assessment of patient indications for therapy.

### System Overview

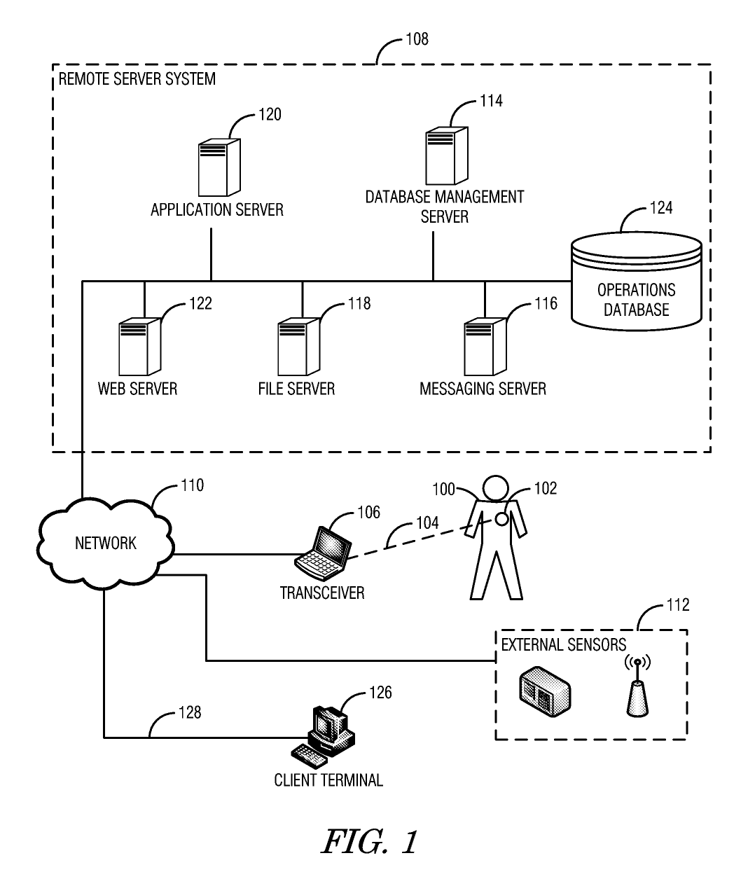

FIG. 1 illustrates portions of a system that enables physician-patient communication. In the example of FIG. 1, a patient 100 is provided with an ambulatory or implantable medical device (IMD) 102. Examples of implantable medical devices include a pacemaker, an implantable cardioverter defibrillator (ICD), a cardiac resynchronization therapy pacemaker (CRT-P), a cardiac resynchronization therapy defibrillator (CRT-D), a pulmonary artery (PA) pressure sensor, a neurostimulation device, a deep brain stimulation device, a cochlear implant or a retinal implant. In some examples, the IMD 102 can be capable of sensing physiological data, deriving physiological measures/correlations, and storing data for later communication or reference. Examples of physiological data include implantable electrograms, surface electrocardiograms, heart rate intervals (e.g., AA, VV, AV or VA intervals), electrogram templates such as for tachyarrhythmia discrimination, pressure (e.g., intracardiac or systemic pressure), oxygen saturation, activity, heart rate variability, heart sounds, impedance, respiration, intrinsic depolarization amplitude, or the like. While only one IMD 102 is illustrated in FIG. 1, it is understood that more than one IMD 102 may be implanted. For example, medical devices that have specific functions can be placed in accordance with their function. In addition, the IMD 102 can be composed of more than one device, with each device having one or more functions.

The IMD 102 can be capable of bidirectional communication using a connection 104 with a computing device 106. A computing device is a device capable of receiving input, processing instructions, storing data, presenting data in a human-readable form, and communicating with other devices. The IMD 102 receives commands from the computing device 106 and can also communicate one or more patient indications to the computing device 106. Examples of patient indications include sensed or derived measurements such as heart rate, heart rate variability, data related to tachyarrhythmia episodes, hemodynamic stability, activity, therapy history, autonomic balance motor trends, electrogram templates for tachy discrimination, heart rate variability trends or templates, or trends, templates, or abstractions derived from sensed physiological data. Patient indications include one or more physiological indications, such as the physiological data described above. The IMD 102 can also communicate one or more device indications to the computing device 106. Examples of device indications include lead/shock impedance, pacing amplitudes, pacing thresholds, or other device metrics. In certain examples, the IMD 102 can communicate sensed physiological signal data to the computing device 106, which can then communicate the signal data to a remote device for processing.

Typically, the computing device 106 can be located in close proximity to the patient 100. The computing device 106 can be attached, coupled, integrated or incorporated with a personal computer or a specialized device, such as a medical device programmer. In an example, the computing device 106 can be a hand-held device. In examples, the computing device 106 can be a specialized device or a personal computer. In an example, the computing device 106 can be adapted to communicate with a remote server system 108. The communication link between the computing device 106 and the remote server system 108 can be made through a computer or telecommunications network 110. The network 110 can include, in various examples, one or more wired or wireless networking such as the Internet, satellite telemetry, cellular telemetry, microwave telemetry, or other long-range communication networks.

In an example, one or more external sensors 112 are adapted to communicate with the computing device 106 or the remote server system 108 and can transmit and receive information, such as sensed data. External sensors 112 can be used to measure patient physiological data, such as temperature (e.g., a thermometer), blood pressure (e.g., a sphygmomanometer), blood characteristics (e.g., glucose level), body weight, physical strength, mental acuity, diet, or heart characteristics. An external sensor 112 can also include one or more environmental sensors. The external sensors 112 can be placed in a variety of geographic locations (in close proximity to patient or distributed throughout a population) and can record non-patient specific characteristics such as, for example, temperature, air quality, humidity, carbon monoxide level, oxygen level, barometric pressure, light intensity, and sound.

External sensors 112 can also include devices that measure subjective data from the patient. Subjective data includes information related to a patient's feelings, perceptions, and/or opinions, as opposed to objective physiological data. For example, the “subjective” devices can measure patient responses to inquiries such as “How do you feel?”, “How is your pain?” and “Does this taste good?” Such a device can also be adapted to present interrogatory questions related to observational data, such as “What color is the sky?” or “Is it sunny outside?” The device can prompt the patient and record responsive data from the patient using visual and/or audible cues. For example, the patient can press coded response buttons or type an appropriate response on a keypad. Alternatively, responsive data can be collected by allowing the patient to speak into a microphone and using speech recognition software to process the response.

In some examples, the remote server system 108 comprises one or more computers, such as a database server 114, a messaging server 116, a file server 118, an application server 120 and a web server 122. The database server 114 can be configured to provide database services to clients, which can be other servers in the remote server system 108. The messaging server 116 can be configured to provide a communication platform for users of the remote server system 108. For example, the messaging server 116 can provide an email communication platform. Other types of messaging, such as short message service (SMS), instant messaging, or paging services. The file server 118 can be used to store documents, images, and other files for the web server 122 or as a general document repository. The application server 120 can provide one or more applications to the web server 122 or provide client-server applications to the client terminals 126. To enable some of these services provided by these servers 114, 116, 118, 120, and 112, the remote server system 108 can include an operations database 124. The operations database 124 can be used for various functions and can be composed of one or more logically or physically distinct databases. The operations database 124 can be used to store clinician data for individual patients, patient populations, patient trials, and the like. In addition, the operations database 124 can be used to store patient data for individual patients, patient populations, patient trials, and the like. For example, the operations database 124 can include a copy of, a portion of, a summary of, or other data from an electronic medical records system. In addition, the operations database 124 can store device information, such as device settings for a particular patient or a group of patients, preferred device settings for a particular clinician or a group of clinicians, device manufacturer information, and the like. In addition, the operations database 124 can be used to store raw, intermediate, or summary data of patient indications along with probabilistic outcomes (e.g., a patient population profile and a corresponding 1-year survival curve).

In an example, one or more client terminals 126 are locally or remotely connected to the remote server system 108 via network 110. The client terminals 112 are communicatively coupled to the remote server system 108 using a connection 128, which can be wired or wireless in various examples. Examples of client terminals 126 can include personal computers, dedicated terminal consoles, handheld devices (e.g., a personal digital assistant (PDA) or cellular telephone), or other specialized devices (e.g., a kiosk). In various examples, one or more users can use a client terminal 126 to access the remote server system 108. For example, a customer service professional can use a client terminal 126 to access records stored in the remote server system 108 to update patient records. As another example, a physician or clinician can use a client terminal 126 to receive or provide patient-related data, such as comments regarding a patient visit, physiological data from a test or collected by a sensor or monitor, therapy history (e.g., IMD shock or pacing therapy), or other physician observations.

In some examples, the IMD 102 can be adapted to store patient data and to use the data to provide tailored therapy. For example, using historical physiological data, an IMD 102 can be able to discriminate between lethal and non-lethal heart rhythms and deliver an appropriate therapy. However, it can be desirable to establish a proper baseline of historical data by collecting a sufficient amount of data in the IMD 102. In some examples, a “learning period” of some time (e.g., thirty days) can be used to establish the baseline for one or more physiological signals. An IMD 102 can, in an example, store a moving window of data of operation, such as a time period equal to the learning period, and can use the information as a baseline indication of the patient's biorhythms or biological events.

Once the baseline is established, then acute and chronic patient conditions can be determined probabilistically. The baseline can be established by using historical patient records or by comparing a patient to a population of patients. In an example, a diagnostic technique uses a patient-based baseline to detect a change in a patient's condition over time. Examples of a diagnostic technique that uses a patient-derived baseline are described in the next section.

In an example, patient diagnostics are automatically collected and stored by the IMD 102. These values can be based on the patient's heart rate or physical activity over a time period (e.g., 24-hour period) and each diagnostic parameter can be saved as a function of the time period. In one example, heart-rate based diagnostics utilize only normal intrinsic beats. For heart rate variability (HRV) patient diagnostics, the average heart rate can be found at each interval within the time period, for example, at each of the 288 five-minute intervals occurring during 24 hours. From these interval values, the minimum heart rate (MinHR), average heart rate (AvgHR), maximum heart rate (MaxHR) and standard deviation of average normal-to-normal (SDANN) values can be calculated and stored. In one example, the IMD 102 computes a HRV Footprint® patient diagnostic that can include a 2-dimensional histogram that counts the number of daily heartbeats occurring at each combination of heart rate (interval between consecutive beats) and beat-to-beat variability (absolute difference between consecutive intervals). Each histogram bin contains the daily total for that combination. The percentage of histogram bins containing one or more counts can be saved each day as the footprint percent (Footprint %). The IMD 102 can also provide an Activity Log® patient diagnostic (Activity %), which can include a general measure of patient activity and can be reported as the percentage of each time period during which the device-based accelerometer signal is above a threshold value.

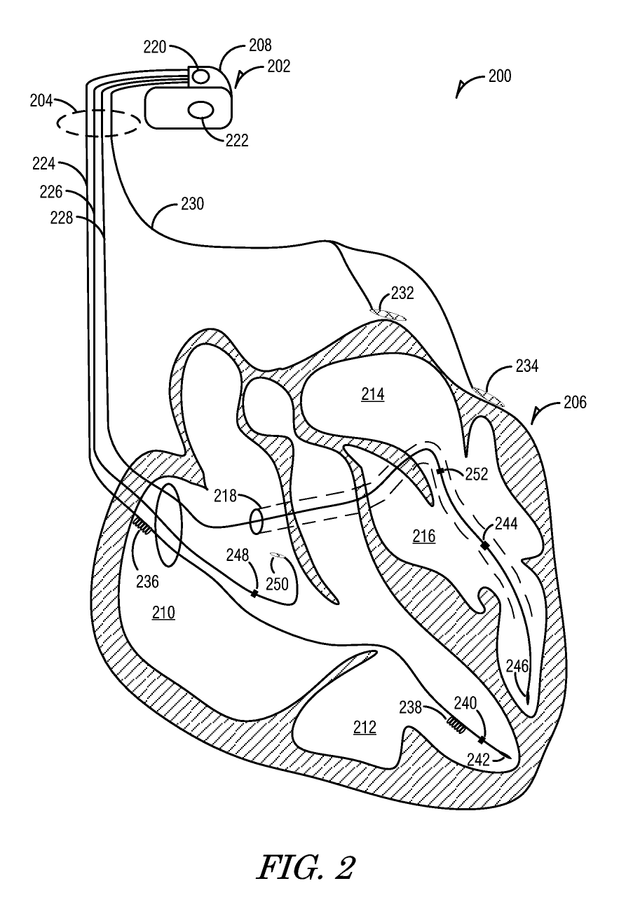

FIG. 2 is a schematic diagram illustrating an implanted medical device (IMD) system 200. The IMD system 200 includes an IMD 202 coupled to a lead system 204 deployed within a heart 206. The lead system 206 can be designed for implantation in a coronary vein for purposes of cardiac resynchronization therapy (CRT). The lead system 206 can be coupled to the IMD 202, which includes a detection/energy delivery system 208 that actively measures and controls the lead system 206 to provide cardiac pacing therapy.

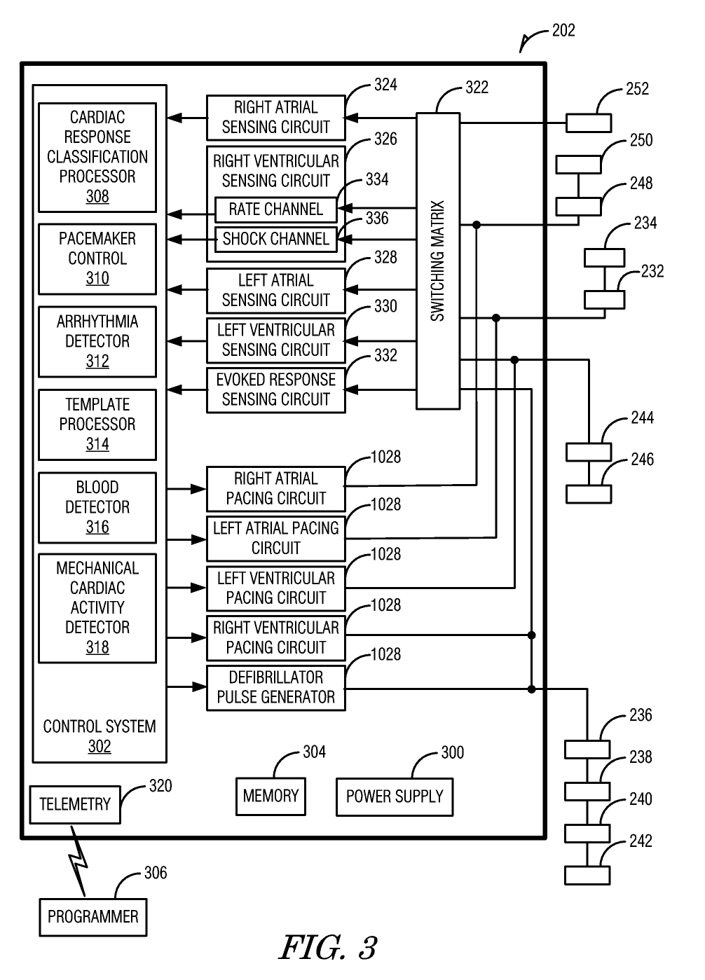

The detector/energy delivery system 208 typically includes a power supply and programmable circuit (e.g., microprocessor) coupled to an analog to digital (A-D) converter (not shown). Various lead system devices, such as electrodes and pressure sensors, can interface to the A-D converter for sensing/data collection. Alternatively, analog conditioning (e.g., filtering) can be applied to sensor signals before interfacing with the A-D converter. The detector/energy delivery system 208 also utilizes an energy delivery system (not shown). The energy delivery system can include charge capacitors and signal conditioning circuitry as is known in the art. The energy system can interface to the programmable circuit through a D-A converter. Components and functionality of the detector/energy delivery system 208 will be further described below with reference to FIG. 3.

The IMD system 200 can be used to implement methods for therapy control based on electromechanical timing. The IMD 202 can be electrically and physically coupled to the lead system 204. The housing and/or header of the IMD 202 can incorporate one or more electrodes 220 and 222 used to provide electrical stimulation energy to the heart 206 and to sense cardiac electrical activity. The IMD 202 can utilize all or a portion of the IMD housing as a can electrode 222. The IMD 202 can include an indifferent electrode 220 positioned, for example, on the header or the housing of the IMD 202. If the IMD 202 includes both a can electrode 222 and an indifferent electrode 220, the electrodes 220 and 222 are electrically isolated from each other.

The heart 206 includes several physiological structures, including a right atrial chamber 210, a right ventricle 212, left atrial chamber 214, and left ventricle 216. The lead system 204 can be implanted into the coronary sinus using various techniques. One such technique, as illustrated in FIG. 2, involves creating an opening in a percutaneous access vessel such as the left subclavian or left cephalic vein. The pacing lead can be guided into the right atrial chamber 210 of the heart via the superior vena cava. From the right atrial chamber 210, the lead system 204 can be sent into the coronary sinus ostium. The ostium is the opening of a coronary sinus 218 into the right atrial chamber 210. The lead system 204 can be guided through the coronary sinus 218 to a coronary vein of the left ventricle 216. A distal end of the lead system 204 can be lodged into the coronary vein.

The lead system 204 can be used to provide pacing signals to the heart 206, detect electric cardiac signals produced by the heart 206, sense blood oxygen saturation, and can also be used to provide electrical energy to the heart 206 under certain predetermined conditions to treat cardiac conditions, such as arrhythmias. The lead system 204 can include one or more electrodes used for pacing, sensing, and/or defibrillation. In the example shown in FIG. 2, the lead system 204 includes an intracardiac right ventricular (RV) lead system 224, an intracardiac right atrial (RA) lead system 226, an intracardiac left ventricular (LV) lead system 228, and an extracardiac left atrial (LA) lead system 230. The lead system 204 can be used for therapy based on electromechanical timing methodologies. Other leads and/or electrodes can additionally or alternatively be used.

The lead system 204 can include intracardiac leads 224, 226, 228 implanted in a human body with portions of the intracardiac leads 224, 226, 228 inserted into the heart 206. The intracardiac leads 224, 226, 228 include one or more electrodes positionable within the heart 206 for sensing electrical activity of the heart 206 and for delivering electrical stimulation energy to the heart 206, for example, pacing pulses and/or defibrillation shocks to treat various arrhythmias.

The lead system 204 can also include one or more extracardiac leads 230 having electrodes, e.g., epicardial electrodes or sensors 232 and 234, positioned at locations outside the heart 206 for sensing and/or pacing one or more heart chambers.

The right ventricular lead system 224 includes an SVC-coil 236, an RV-coil 238, an RV-tip electrode 240, and an RV-ring electrode 242. The right ventricular lead system 224 extends through the right atrium 210 and into the right ventricle 212. In particular, the RV-tip electrode 240, RV-ring electrode 242, and RV-coil electrode 238 are positioned at appropriate locations within the right ventricle 212 for sensing and delivering electrical stimulation pulses to the heart. The SVC-coil 236 can be positioned at an appropriate location within the right atrium chamber 210 of the heart 206 or a major vein leading to the right atrial chamber 210.

In one configuration, the RV-tip electrode 240 referenced to the can electrode 222 can be used to implement unipolar pacing and/or sensing in the right ventricle 212. Bipolar pacing and/or sensing in the right ventricle 212 can be implemented using the RV-tip 240 and RV-ring 242 electrodes. In yet another configuration, the RV-ring 242 electrode can optionally be omitted and bipolar pacing and/or sensing can be accomplished using the RV-tip electrode 240 and the RV-coil 238, for example. The right ventricular lead system 224 can be configured as an integrated bipolar pace/shock lead. The RV-coil 238 and the SVC-coil 236 are defibrillation electrodes.

The left ventricular lead 228 includes an LV distal electrode 244 and an LV proximal electrode 246 located at appropriate locations in or about the left ventricle 216 for pacing and/or sensing the left ventricle 216. The left ventricular lead 228 can be guided into the right atrium 210 of the heart via the superior vena cava. From the right atrium 210, the left ventricular lead 228 can be deployed into the coronary sinus ostium, the opening of the coronary sinus 218. The left ventricle lead 228 can be guided through the coronary sinus 218 to a coronary vein of the left ventricle 216. This vein can be used as an access pathway for leads to reach the surfaces of the left ventricle 216 which are not directly accessible from the right side of the heart, and to sense blood oxygen levels in the blood leaving the myocardium. Lead placement for the left ventricular lead 228 can be achieved via subclavian vein access and a preformed guiding catheter for insertion of the LV electrodes 244 and 246 proximate to the left ventricle.

Unipolar pacing and/or sensing in the left ventricle 216 can be implemented, for example, by using the LV distal electrode 244 referenced to the can electrode 222. The LV distal electrode 244 and the LV proximal electrode 246 can be used together as bipolar sense and/or pace electrodes for the left ventricle 216. The left ventricular lead 228 and the right ventricular lead 224, in conjunction with the IMD 202, can be used to provide cardiac resynchronization therapy such that the ventricles of the heart are paced substantially simultaneously, or in phased sequence, to provide enhanced cardiac pumping efficiency for patients suffering from various symptoms of heart failure.

The right atrial lead 226 includes a RA-tip electrode 248 and an RA-ring electrode 250 positioned at appropriate locations in the right atrium 210 for sensing and pacing the right atrium 210. In one configuration, the RA-tip electrode 248 referenced to the can electrode 222, for example, can be used to provide unipolar pacing and/or sensing in the right atrium 210. In another configuration, the RA-tip electrode 248 and the RA-ring electrode 250 can be used to provide bipolar pacing and/or sensing.

The left ventricular lead 228 can include a pressure transducer 252. The pressure transducer 252 can be a micro-electrical-mechanical system (MEMS), for example. MEMS technology uses semiconductor techniques to build microscopic mechanical devices in silicon or similar materials. The pressure transducer 252 can include a micromachined capacitive or piezoresistive transducer exposed to the bloodstream. Other pressure transducer technologies, such as resistive strain gages, are known in the art and can also be employed as a pressure transducer 252. The pressure transducer 252 can be coupled to one or more conductors disposed along the length of the left ventricular lead 228. The pressure transducer 252 can be integrated with the left ventricular lead 228. Transducers such as the pressure transducer 252 can be used to determine pulmonary arterial (PA) pressure information useful for determining electromechanical delay (EMD) information.

FIG. 3 is a block diagram illustrating an implantable medical device (IMD) 202. The IMD 202 can be suitable for therapy control based on electromechanical timing. The IMD 202 can be divided into functional blocks. It will be understood by those skilled in the art that there exist many possible configurations in which these functional blocks can be arranged and that the example illustrated in FIG. 3 is but one possible arrangement. In addition, although the IMD 202 contemplates the use of a programmable microprocessor-based logic circuit, other circuit implementations can be used.

The IMD 202 includes circuitry for receiving cardiac signals from a heart and delivering electrical stimulation energy to the heart in the form of pacing pulses and/or defibrillation shocks. In one embodiment, the circuitry of the PIMD 900 can be encased and hermetically sealed in a housing suitable for implanting in a human body. Power to the IMD 202 can be supplied by an electrochemical battery 300. A connector block (not shown) can be attached to the housing of the IMD 202 to provide for the physical and electrical attachment of the lead system conductors to the circuitry of the IMD 202.

The IMD 202 can be a programmable microprocessor-based system, including a control system 302 and a memory 304. The memory 304 can store parameters for various pacing, defibrillation, and sensing modes, along with other parameters. Further, the memory 304 can store data indicative of signals received by other components of the IMD 202. The memory 302 can be used, for example, for storing historical EMT/EMD information, blood oxygen levels, blood flow information, perfusion information, heart sounds, heart movement, EGM, and/or therapy data. The historical data storage can include, for example, data obtained from long-term patient monitoring used for trending or other diagnostic purposes. Historical data, as well as other information, can be transmitted to an external programmer 306 as needed or desired.

The control system 302 and memory 304 can cooperate with other components of the IMD 202 to control the operations of the IMD 202. The control system 302 incorporates a cardiac response classification processor 308 for classifying cardiac responses to pacing stimulation. The control system 302 can include additional functional components including a pacemaker control circuit 310, an arrhythmia detector 312, a template processor 314 for cardiac signal morphology analysis, and a blood detector 316 configured to determine blood perfusion, blood flow, and/or blood pressure based on one or more sensors. The control system 302 can optionally, or additionally, include a mechanical cardiac activity detector 318 configured to detect mechanical cardiac activity using sensor information from, for example, an accelerometer, a microphone, a pressure transducer, impedance sensors, or other motion or sound sensing arrangements.

A telemetry circuitry 320 can be implemented to provide communications between the IMD 202 and the external programmer 306. In an example, the telemetry circuitry 320 and the programmer 306 communicate using a wire loop antenna and a radio frequency telemetric link, as is known in the art, to receive and transmit signals and data. In this manner, programming commands and other information can be transferred to the control system 302 from the programmer 306 during and after implant. In addition, stored cardiac data pertaining to EMT/EMD, capture threshold, capture detection and/or cardiac response classification, for example, along with other data, can be transferred to the programmer 306 from the IMD 202.

The telemetry circuitry 320 can also allow the IMD 202 to communicate with one or more receiving devices or systems situated external to the IMD 202. By way of example, the IMD 202 can communicate with a patient-worn, portable or bedside communication system via the telemetry circuitry 320. In one configuration, one or more physiologic or non-physiologic sensors (subcutaneous, cutaneous, or external of patient) can be equipped with a short-range wireless communication interface, such as an interface conforming to a known communications standard, such as Bluetooth or IEEE 802 standards. Data acquired by such sensors can be communicated to the IMD 202 via the telemetry circuitry 320. It is noted that physiologic or non-physiologic sensors equipped with wireless transmitters or transceivers can communicate with a receiving system external of the patient. The external sensors in communication with the IMD 202 can be used to determine electromechanical timing and/or delays in accordance with embodiments of the present invention.

As illustrated in FIG. 2 and reproduced in FIG. 3, one or more leads can be coupled to the IMD 202 to provide for sensing and therapy. In the example illustrated in FIG. 3, the electrodes include: RA-tip 248, RA-ring 250, RV-tip 240, RV-ring 242, RV-coil 238, SVC-coil 236, LV distal electrode 244, LV proximal electrode 246, LA distal electrode 234, LA proximal electrode 232, indifferent electrode 220, and can electrode 222 are coupled through a switch matrix 322 to sensing circuits 324, 326, 328, 330, and 332.

A right atrial sensing circuit 324 serves to detect and amplify electrical signals from the right atrium of the heart. Bipolar sensing in the right atrium can be implemented, for example, by sensing voltages developed between the RA-tip 248 and the RA-ring 250. Unipolar sensing can be implemented, for example, by sensing voltages developed between the RA-tip 248 and the can electrode 222. Outputs from the right atrial sensing circuit 324 are coupled to the control system 302.

A right ventricular sensing circuit 326 serves to detect and amplify electrical signals from the right ventricle of the heart. The right ventricular sensing circuit 326 can include, for example, a right ventricular rate channel 334 and a right ventricular shock channel 336. Right ventricular cardiac signals sensed through use of the RV-tip 240 electrode are right ventricular near-field signals and are denoted RV rate channel signals. A bipolar RV rate channel signal can be sensed as a voltage developed between the RV-tip 240 and the RV-ring 242. Alternatively, bipolar sensing in the right ventricle can be implemented using the RV-tip electrode 240 and the RV-coil 238. Unipolar rate channel sensing in the right ventricle can be implemented, for example, by sensing voltages developed between the RV-tip 240 and the can electrode 222.

Right ventricular cardiac signals sensed through use of the RV-coil electrode 238 are far-field signals, also referred to as RV morphology or RV shock channel signals. More particularly, a right ventricular shock channel signal can be detected as a voltage developed between the RV-coil 238 and the SVC-coil 236. A right ventricular shock channel signal can also be detected as a voltage developed between the RV-coil 238 and the can electrode 222. In another configuration the can electrode 222 and the SVC-coil electrode 236 can be electrically shorted and a RV shock channel signal can be detected as the voltage developed between the RV-coil 238 and the can electrode 222/SVC-coil 236 combination.

Left atrial cardiac signals can be sensed through the use of one or more left atrial electrodes 232, 234, which can be configured as epicardial electrodes. A left atrial sensing circuit 328 serves to detect and amplify electrical signals from the left atrium of the heart. Bipolar sensing and/or pacing in the left atrium can be implemented, for example, using the LA distal electrode 234 and the LA proximal electrode 232. Unipolar sensing and/or pacing of the left atrium can be accomplished, for example, using the LA distal electrode 234 to can electrode 222 or the LA proximal electrode 232 to can electrode 222.

A left ventricular sensing circuit 330 serves to detect and amplify electrical signals from the left ventricle of the heart. Bipolar sensing in the left ventricle can be implemented, for example, by sensing voltages developed between the LV distal electrode 244 and the LV proximal electrode 246. Unipolar sensing can be implemented, for example, by sensing voltages developed between the LV distal electrode 244 or the LV proximal electrode 246 and the can electrode 222. Optionally, an LV coil electrode (not shown) can be inserted into the patient's cardiac vasculature, e.g., the coronary sinus, adjacent the left heart. Signals detected using combinations of the LV electrodes, 244, 246, LV coil electrode (not shown), and/or can electrode 222 can be sensed and amplified by the left ventricular sensing circuitry 330. The output of the left ventricular sensing circuit 330 can be coupled to the control system 302.

### Example Operations

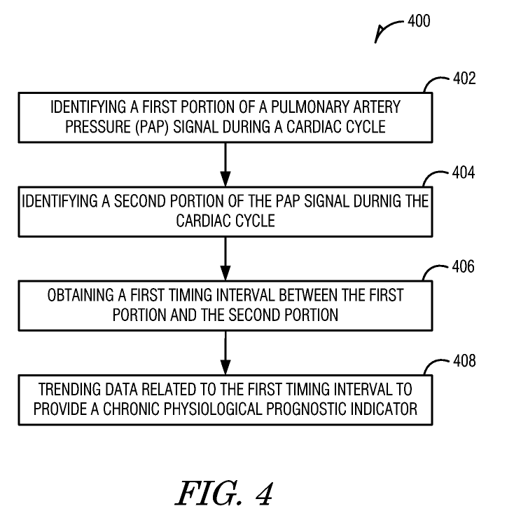

FIG. 4 is a flow chart illustrating a method 400 for obtaining a prognostic indicator. At 402, a first portion of a pulmonary artery pressure (PAP) signal can be identified during a cardiac cycle. In an example, to identify the first portion of the PAP signal, a feature of an intrinsic electrical cardiac signal during the cardiac cycle can be identified. The feature of the intrinsic electrical cardiac signal can be correlated with a position on the PAP signal to identify the first portion of the PAP signal. In an example, a feature indicative of the start of depolarization of ventricles can be identified as the feature of the intrinsic electrical cardiac signal during the cardiac cycle.

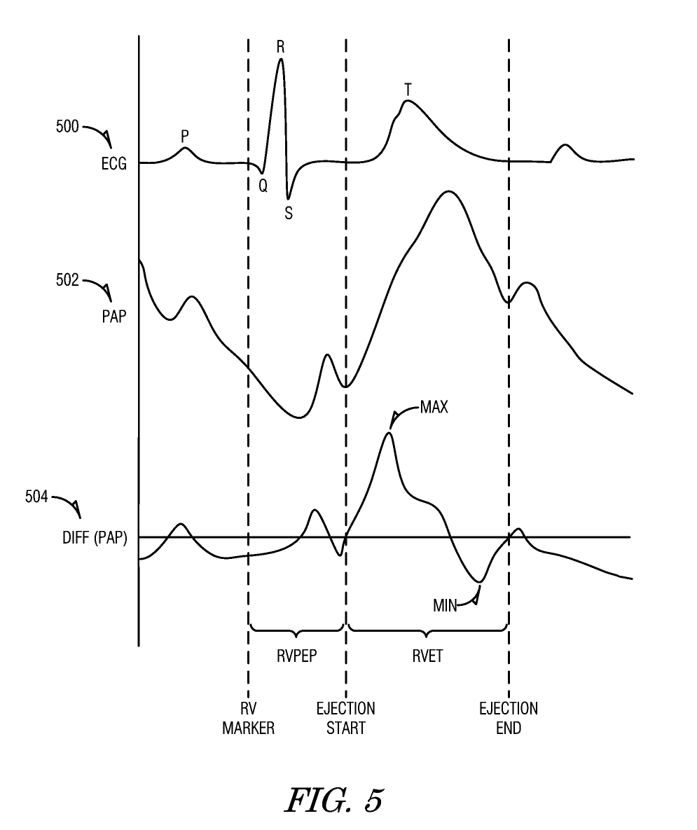

Referring to FIG. 5, three waveforms are illustrated including an electrocardiogram (ECG) signal 500, a pulmonary artery pressure (PAP) signal 502, and a first derivative of the PAP signal 504. The ECG signal 500 can be obtained from an external or internal sensor. For example, the ECG signal 500 can be obtained by a noninvasive recording produced by an electrocardiographic device that implements one or ore skin electrodes. As another example, the ECG signal 500 can be obtained from a chronically implanted device, such as IMD 202 (FIG. 2). The PAP signal 502 can be directly obtained or indirectly derived. For example, a sensor can be coupled to a pulmonary artery catheter. Vascular pressures can be measured with one or more transducers. Cardiac output can be measured using a thermodilution technique. The ECG signal 500 and the PAP signal 504 illustrated in FIG. 5 can represent averaged signals. For example, five cardiac cycles can be captured by the electrocardiographic device and aligned using morphological analysis. Abnormal or anomalous signals can be discarded to obtain a good representation of an average signal. In addition, the signals can be upsampled to provide additional resolution, as is known in the art.

One method to identify a feature indicative of the start of depolarization of ventricles can be to review the ECG signal 500. For example, the first feature of the intrinsic electrical cardiac signal (e.g., the ECG signal 500) can be the onset of the Q-wave. This is also referred to as an “RV marker” signifying the beginning of the right ventricular action. When correlating the RV marker with the PAP signal 504, a first portion of the PAP signal 504 can be identified.

Referring again to FIG. 4, at 404, a second portion of the PAP signal can be identified during the cardiac cycle. The second portion can be any morphological feature of the PAP curve.

In an example, to identify the first portion of the PAP signal, a start of a right ventricle pre-ejection period (RVPEP) can be identified. In an example, this can be performed by analyzing the electrical signal and correlating the electrical signal with a mechanical signal. However, the disclosure is not limited to this mechanism as other mechanisms to identify the start of the RVPEP can be used. To identify the second portion of the PAP signal, an end of the right ventricle pre-ejection period can be identified. In an example, the end of the RVPEP can be identified by finding the start of the RVET.

Referring again to FIG. 5, the start of the RVPEP can be found by using the RV marker, as previously discussed. The end of the RVPEP can be found by identifying the start of ejection. In FIG. 5, this point can be found at the dip before the rapid upstroke of the PAP curve that occurs after the QRS complex during the ST segment. Using the first derivative PAP curve 504, the zero crossing can readily be identified as the ejection start.

In another example, the first and second portions are identified by respectively identifying a start of a right ventricle ejection time and identifying an end of the right ventricle ejection time. Ejection time can be determined by analyzing the PAP curve 502, in particular from the start of the rapid upstroke to the dichrotic notch. Again, using the first differential PAP curve, a zero crossing can be identified for both the start and the end of the ejection time. In this instance, the zero crossing that occurs before the Max can be used as the ejection start time and the zero crossing that occurs after the Min can be used as the ejection end time. To obtain the ejection time, the ejection start time can be subtracted from the ejection end time.

Referring again to FIG. 4, at 406, a first timing interval between the first portion and the second portion can be obtained. As described above, the first timing interval can be the RVPEP or the RVET. Other intervals can be identified and used, such as an interval from the start of ejection to the period of maximum pulmonary artery pressure, or an interval from the period of maximum pulmonary artery pressure to the end of ejection.

At 408, data related to the first timing interval can be trended to provide a chronic physiological prognostic indicator. Data trending can be performed using various forms of regression analysis, such as linear regression, non-linear regression, least squares, Bayesian methods, quintile regression, or nonparametric regression.

In an example, a third portion of the PAP signal during the cardiac cycle can be identified. In addition, a fourth portion of the PAP signal during the cardiac cycle can be identified. A second timing interval between the third portion and the fourth portion can be obtained. Data can be then trended using a function of the first and second timing intervals to provide the chronic physiological prognostic indicator. It is understood that “first,” “second,” “third,” and “fourth” are merely labels used for convenience and that in some examples a “first portion” can refer to the same portion labeled a “second portion.”

In an example, the first and second intervals can be trended by first calculating a ratio of the first interval to the second interval to provide an estimated right ventricle ejection fraction (RVEF) and then trending the estimated RVEF. It can be shown that there is a strong correlation between the ratio of RVPEP/RVET and the RVEF. In an example, the RVEF can be estimated by taking the ratio of the RVPEP to the RVET The RVEF can then be estimated using regression analysis.

In an example, the chronic physiological prognostic indicator can be compared to a threshold value to provide a result. The result can be then used to identify a treatment. In a study, it was found that an RVEF of 24% was predictive of mortality. That is, patients with an RVEF under 24% were found to be at a much higher risk of mortality than patients with an RVEF over 24%. In this case, a threshold value of 24% can be used to classify patients and identify patients at a higher risk.

In another study, it was found that patients with a high PAP and a low RVEF were at a much higher risk than those with a normal PAP and only slightly depressed RVEF. In addition, the patients with high PAP and low RVEF were found to be at a higher risk than those with a high PAP and slightly depressed RVEF. In this case, a threshold value based on two factors, PAP and RVEF, can be used. In an example, the threshold value can be an indexed value that incorporates the PAP and RVEF values. For instance, a right ventricular performance index (RVPI) can be calculated with the following function: RVPI=RVEF/PAP. So RVPI is directly proportional to RVEF, that is, as RVEF goes up, the RVPI is a higher value. Also, the RVPI is inversely proportional to the PAP, so as PAP decreases, the RVPI increases.

In an example, a plurality of chronic physiological prognostic indicators are obtained over time. The plurality of chronic physiological prognostic indicators are trended to provide a result and a treatment can be identified in response to the result. By trending the prognostic indicators, a patient's chronic disposition can be observed.

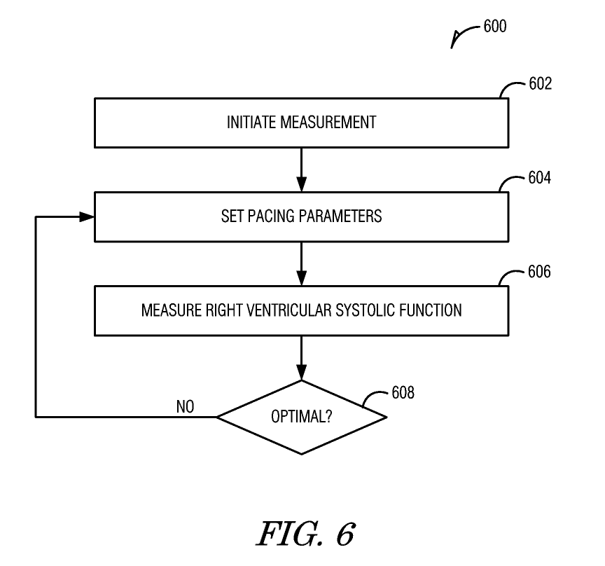

FIG. 6 is a flow chart illustrating a method 600 for managing pacing parameters. At 602, one or more measurements are initiated. Measurements can include the number of events that have occurred since the previous programming checkup. For example, the pre-ejection period, ejection time, or the RVEF can be measured during an office visit and compared to previously-obtained measurements. At 604, one or more pacing parameters are set. Pacing parameters include, but are not limited to, parameters for various pacing, defibrillation, and sensing modes. For example, in the context of a pacing device, various parameters such as pacing amplitude, pacing rate, and pulse width can be configured or adjusted by a clinician or other care provider. At 606, right ventricular (RV) systolic function can be measured. In an example, measurement can be performed using the method 400, as described above. At 608, it can be determined whether the RV systolic function is optimal or improving. If the RV systolic function is not optimal, or at least improving, then the method 600 returns to block 604 to modify pacing parameters and the method 600 flows through to evaluate the RV systolic function again at block 606. While pacing parameters are described with respect to FIG. 6, it is understood that any modification to a patient's device can be performed at block 604, such as lead repositioning or electrical repositioning, in an effort to optimize RV systolic function. As an example, multiple sets of pacing parameters can be developed and used over time, tracking the effectiveness of each set as it is used and modifying pacing parameters to increase or optimize RVEF. As another example, pacing parameters or other therapy modifications can be used to lower or minimize PEP, or raise or maximize RVET, each thereby having the effect of improving or raising RVEF.

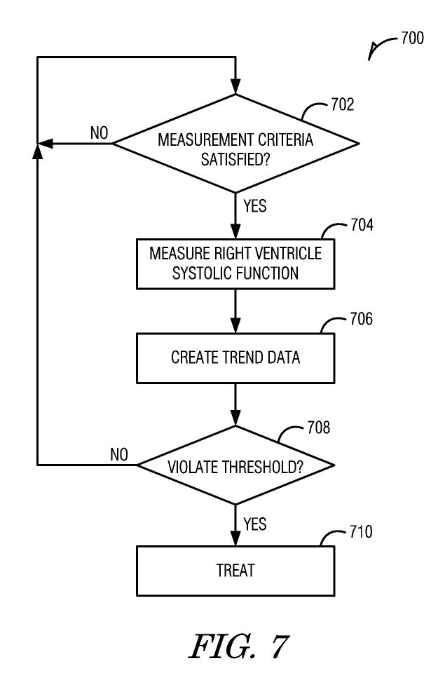

FIG. 7 is a flow chart illustrating a method 700 for patient management. At 702, it can be determined whether one or more measurement criteria are satisfied. For example, one or more beat selection parameters are used to filter a signal (e.g., an ECG signal or PAP signal). Beat selection parameters include, but are not limited to, activity, posture, and respiration. Using such parameters, two or more beats are selected that are obtained under similar conditions (e.g., when the patient is upright and at rest).

After the measurement criteria are satisfied, RV systolic function can be measured at 704. RV systolic function can be a measurement of one or more RV STIs, either alone or in combination, such as an RVET, RVEF or RVPI.

At 706, trend data of the RV systolic function measurements are created. In an example, other sensor data can be used as at least a part of the trend data. For example, while trending RVEF, left ventricular (LV) performance can also be trended. Measuring both LV performance, such as by left ventricular ejection fraction (LVEF), in combination with RVEF can give a physician a better understanding of a patient's ongoing condition.

At 708, it can be determined whether the data trend violates a threshold. For example, a sharp decrease in RVEF with a corresponding increase in PAP can be recognized as a critical situation. Threshold values can be based on the patient's own history, a patient population, or a combination of measurements. In an example, an RVEF of 24% can be used as a threshold value. Patients with RVEF's lower than 24% are considered at higher risk for severe health complications or mortality. Although RVEF is discussed, other measurements could be thresholded individually or in combination with RVEF, such as RVPEP or RVET.

At 710, when a threshold is violated, a treatment can be initiated or performed. For example, an office visit can be recommended, where a physician can modify one or more pacing parameters. As another example, pacing parameters can adaptively change based on the trended data in view of the threshold.

### Example Machine Architecture

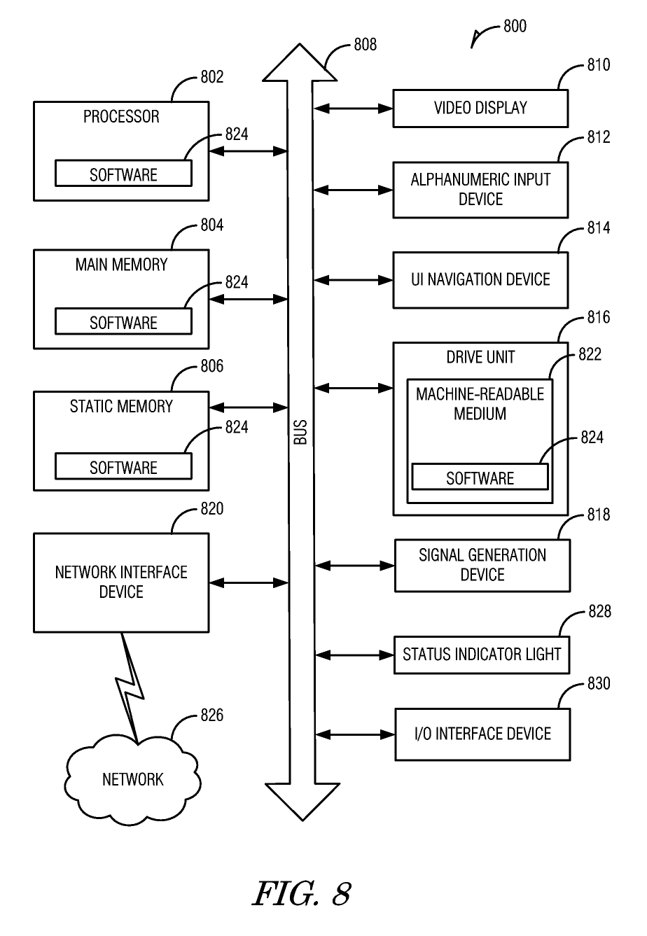

FIG. 8 is a block diagram of machine in the example form of a computer system 800 within which a set of instructions, for causing the machine to perform any one or more of the methodologies discussed herein may be executed. In alternative examples, the machine operates as a standalone device or may be connected (e.g., networked) to other machines. In a networked deployment, the machine may operate in the capacity of a server or a client machine in server-client network environment, or as a peer machine in a peer-to-peer (or distributed) network environment. The machine may be a server computer, a client computer, a personal computer (PC), a tablet PC, a Personal Digital Assistant (PDA), a cellular telephone, a web appliance, a device programmer, a repeater, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The example computer system 800 includes a processor 802 (e.g., a central processing unit (CPU) a graphics processing unit (GPU) or both), a main memory 804 and a static memory 806, which communicate with each other via a bus 808. The computer system 800 may further include a video display 810 (e.g., a liquid crystal display (LCD) or a cathode ray tube (CRT)). The computer system 800 also includes an alphanumeric input device 812 (e.g., a keyboard), a user interface navigation device 814 (e.g., a mouse), a disk drive unit 816, a signal generation device 818 (e.g., a speaker) and a network interface device 820.

The disk drive unit 816 includes a machine-readable medium 822 on which is stored one or more sets of instructions (e.g., software 824) embodying any one or more of the methodologies or functions described herein. The software 824 may also reside, completely or at least partially, within the main memory 804 and/or within the processor 802 during execution thereof by the computer system 800, the main memory 804 and the processor 802 also constituting machine-readable media. The software 824 may further be transmitted or received over a network 826 via the network interface device 820.

While the machine-readable medium 822 is shown in an example embodiment to be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies of the present invention. The term “machine-readable medium” shall accordingly be taken to include, but not be limited to, tangible media, such as solid-state memories, optical, and magnetic media.

### Additional Notes

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown and described. The above description is intended to be illustrative, and not restrictive. For example, the above-described examples (or one or more aspects thereof) may be used in combination with each other. Other embodiments can be used, such as those identified by one of ordinary skill in the art upon review of the above description.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. A system, comprising:
a machine-readable media; and
one or more processors communicatively coupled to the machine-readable media, the machine-readable media including instructions, which when executed by the one or more processors, cause the one or more processors to:
identify a first portion of a pulmonary artery pressure (PAP) signal during a cardiac cycle;
identify a second portion of the PAP signal during the cardiac cycle;
obtain a first timing interval between the first portion and the second portion; and
trend data related to the first timing interval to provide a chronic physiological prognostic indicator.

2. The system of claim 1, wherein the instructions to identify the first portion of the PAP signal during the cardiac cycle further comprises instructions to:
identify a feature of an intrinsic electrical cardiac signal during the cardiac cycle; and
correlate the feature of the intrinsic electrical cardiac signal with a position on the PAP signal to identify the first portion of the PAP signal.

3. The system of claim 2, wherein the instructions to identify the feature of the intrinsic electrical cardiac signal during the cardiac cycle further comprises instructions to identify a feature indicative of the start of depolarization of ventricles.

4. The system of claim 1, wherein the instructions to identify the first portion of the PAP signal during the cardiac cycle further comprises instructions to identify a start of a right ventricle pre-ejection period, and wherein the instructions to identify the second portion of the PAP signal during the cardiac cycle further comprises the instructions to identify an end of the right ventricle pre-ejection period.

5. The system of claim 1, wherein the instructions to identify the first portion of the PAP signal during the cardiac cycle further comprises the instructions to identify a start of a right ventricle ejection time, and wherein the instructions to identify the second portion of the PAP signal during the cardiac cycle further comprises the instructions to identify an end of the right ventricle ejection time.

6. The system of claim 1, wherein the machine-readable media includes instructions, which when executed by the one or more processors, cause the one or more processors to:
identify a third portion of the PAP signal during the cardiac cycle;
identify a fourth portion of the PAP signal during the cardiac cycle; and
obtain a second timing interval between the third portion and the fourth portion, and wherein the instructions to trend data related to the timing interval further comprises the instructions to trend a function of the first and second timing intervals to provide the chronic physiological prognostic indicator.

7. The system of claim 6, wherein the instructions to trend the function of the first and second timing intervals further comprises instructions to:
calculate a ratio of the first interval to the second interval to provide an estimated right ventricle ejection fraction (RVEF); and
trend the estimated RVEF.

8. The system of claim 1, wherein the machine-readable media includes instructions, which when executed by the one or more processors, cause the one or more processors to:
compare the chronic physiological prognostic indicator to a threshold value to provide a result; and
identify a treatment in response to the result.

9. The system of claim 1, wherein the machine-readable media includes instructions, which when executed by the one or more processors, cause the one or more processors to:
obtain a plurality of chronic physiological prognostic indicators over time;
trend the plurality of chronic physiological prognostic indicators to provide a result; and
identify a treatment in response to the result.

10. The system of claim 1, wherein the machine-readable media includes instructions, which when executed by the one or more processors, cause the one or more processors to:
determine systolic function based on the chronic physiological prognostic indicator;
determine whether the systolic function is improving; and
adjust one or more pacing parameters when the systolic function is not improving, wherein the pacing parameters are to configure an implanted cardiac device.

11. The system of claim 1, comprising:
a chronically implanted device having one or more sensors to acquire the PAP signal during the cardiac cycle.

12. A method comprising:
identifying a first portion of a pulmonary artery pressure (PAP) signal during a cardiac cycle;
identifying a second portion of the PAP signal during the cardiac cycle; obtaining a first timing interval between the first portion and the second portion; and
trending data related to the first timing interval to provide a chronic physiological prognostic indicator.

13. The method of claim 12, wherein identifying the first portion of the PAP signal during the cardiac cycle comprises:
identifying a feature of an intrinsic electrical cardiac signal during the cardiac cycle; and
correlating the feature of the intrinsic electrical cardiac signal with a position on the PAP signal to identify the first portion of the PAP signal.

14. The method of claim 13, wherein identifying the feature of the intrinsic electrical cardiac signal during the cardiac cycle comprises identifying a feature indicative of the start of depolarization of ventricles.

15. The method of claim 12, wherein identifying the first portion of the PAP signal during the cardiac cycle comprises identifying a start of a right ventricle pre-ejection period, and wherein identifying the second portion of the PAP signal during the cardiac cycle comprises identifying an end of the right ventricle pre-ejection period.

16. The method of claim 12, wherein identifying the first portion of the PAP signal during the cardiac cycle comprises identifying a start of a right ventricle ejection time, and wherein identifying the second portion of the PAP signal during the cardiac cycle comprises identifying an end of the right ventricle ejection time.

17. The method of claim 12, comprising:
identifying a third portion of the PAP signal during the cardiac cycle;
identifying a fourth portion of the PAP signal during the cardiac cycle; and
obtaining a second timing interval between the third portion and the fourth portion, and wherein trending data related to the timing interval comprises trending a function of the first and second timing intervals to provide the chronic physiological prognostic indicator.

18. The method of claim 17, wherein trending the function of the first and second timing intervals comprises:
calculating a ratio of the first interval to the second interval to provide an estimated right ventricle ejection fraction (RVEF); and
trending the estimated RVEF.

19. The method of claim 12, comprising:
comparing the chronic physiological prognostic indicator to a threshold value to provide a result; and
identifying a treatment in response to the result.

20. A system comprising:
a physiological sensor to obtain a pulmonary artery pressure (PAP) signal;
a memory device to store the PAP signal;
means for identifying a first portion of the PAP signal during a cardiac cycle;
means for identifying a second portion of the PAP signal during the cardiac cycle;
means for obtaining a first timing interval between the first portion and the second portion; and
means for trending data related to the first timing interval to provide a chronic physiological prognostic indicator.
